(19) United States
(12) Patent Application Publication
(10) Pub. No.: US 2004/0082932 A1
(43) Pub. Date: Apr. 29, 2004

(54) Absorbent garment having a waist belt attachment system

(76) Inventor: Nels Lauritzen, Piscataway, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/691,719

(22) Filed: Oct. 22, 2003

Related U.S. Application Data
(62) Division of application No. 09/464,582, filed on Dec. 16, 1999.

(51) Int. Cl. A61F 13/15

(52) U.S. Cl. 604/392

(57) ABSTRACT

An absorbent garment, such as an adult incontinent brief, having a waist belt attached to a suspension sling. The waist belt, in association with an attachment system, is intended to encircle and fasten about the waist of a wearer, allowing the suspension sling to be securely suspended in the wearer's crotch region.

### FIELD OF THE INVENTION

The present invention relates to absorbent garments that may be worn about a wearer's lower torso, such as adult incontinence garments, and in particular to absorbent garments having a waist belt for encircling a wearer's waist.

### BACKGROUND OF THE INVENTION

Garments of the general type described are well known. In particular, disposable garments and incontinent garments are widely described in the patent literature and elsewhere. Among such publications are patents that relate to classes of garments that are provided with various suspensions and/or attaching means. For example, Ahr. U.S. Pat. No. 4,909,802, teaches a disposable absorbent garment having an integral belt segment on each side of the garment. The integral belt segments, in association with receiving or attachment means, are intended to hold or suspend the garment about the lower torso of the wearer.

A similar product is disclosed in Gipson, U.S. Pat. No. 4,964,860. In this product, a reusable belt is detachable from a disposable absorbent assembly. The belt has indicia to mark its longitudinal centerline as a target for attaching the first end of the disposable absorbent assembly. In order to put the Gipson product on her own torso, the user would attach one end of the disposable absorbent assembly to the belt proximate the indicia. Next, the article is placed about her waist with the absorbent assembly hanging behind her and the belt is drawn about and encircles the torso. The user will then secure the ends of the belt together. She can then reach down between her legs to retrieve the second end of the disposable absorbent assembly to attach it to the belt in front of her. This requires the user to handle an awkward assembly hanging down behind her where it may become soiled prior to being secured about her waist. In order to avoid this sequence additional steps may be required.

Garments of she type described in the prior art, as well as those which have been available commercially, suffer from one or more of the following deficiencies: they are uncomfortable in that the elastic strips and buttons used to secure them about the body tend to irritate the wearer; they are size-dependent and unable to accommodate a variety of wearers; they tend to sag in use resulting in significant leakage with its attendant psychological effects; they are expensive to manufacture; they are not readily refastenable, thereby inhibiting a parent, an incontinent, or a person in charge of a incontinent from opening the garment and examining for wetness or from easily lowering and then refastening he garment when using bathroom facilities; they are overly bulky in the front where the belt portions fasten to one another and to the garment; and they are difficult to use for people who have limited dexterity and range of motion.

Therefore, it is an object of the present invention to provide a combination of an absorbent garment and a waist belt attachment system that overcomes, to a great degree, the deficiencies in the prior art structures. It is another object of the present invention to provide an absorbent structure that is pulled up to the user's body to avoid excessive sag-induced leakage. It is yet another object of the present invention to provide a product line that requires fewer product sizes and/or shapes to accommodate the wide variety of sizes and shapes of users of the product. It is an additional object of a portion of the present invention to provide an absorbent garment having a single integral belt for use as a waist belt attachment system. It is a further object of the present invention to provide an absorbent garment that may easily be used by people having limited dexterity and range of motion.

While the subject technology has application on all drawer-like garments (i.e., garments that encircle the waist and are drawn through the crotch of a wearer), it has particular application on drawer-like garments in which the sides of the crotch portion do not meet along the sides of the wearer when worn, as is often the case with adult size incontinence garments.

### SUMMARY OF THE INVENTION

In accordance with the present invention, an absorbent garment, such as an adult incontinence garment, is provided with a suspension sling and a waist belt segment attached to the suspension sling. The suspension sling comprises an absorbent structure having a garment surface, a body surface, and side edges; a liquid impervious backsheet positioned adjacent the garment surface of the absorbent structure; and a liquid pervious topsheet positioned adjacent the body surface of the absorbent structure.

In one preferred embodiment, the waist belt attachment system comprises a single integral belt segment and a belt attachment system for use in association with the integral belt segment. A first end of the integral belt segment is joined to a proximal end of the suspension sling to form an “L-shape”. Each end of the integral belt segment is also provided with belt attachment system components which are intended to cooperate with each other to fasten the belt about the waist of the wearer. Further, a central portion of the integral belt segment and he distal end of the suspension sling are provided with sling attachment system components to fasten the distal end of the sling to the integral belt when the garment is in place around a wearer's torso.

In another preferred embodiment, the waist belt has a first belt end, a second belt end, a first sling attachment location proximate the first belt end, and a second sling attachment location disposed between the first and second belt ends. The suspension sling is releasably attachable to the sling attachment locations. After use, the suspension sling is removable from the waist belt for disposal, and another suspension sling may be releasably attached to the sling attachment locations.

The embodiments of this invention successfully achieve one or more of the objects of the invention identified above.

### BRIEF DESCRIPTION OF THE DRAWINGS

FIG. 1 is a perspective view of a disposable absorbent garment embodiment of the present invention.

FIG. 1a is a cross-section of the waist belt of FIG. 1 taken across line 1a-1a.

FIG. 2 is a perspective view of the disposable absorbent garment embodiment of FIG. 1 showing the integral waist belt in a position as it would encircle a wearer's waist.

FIG. 3 is a perspective view of the disposable absorbent garment embodiment of FIG. 2 further showing the suspension sling in a position as it would be drawn between a wearer's legs.

FIG. 4 is a perspective view of a disposable absorbent garment embodiment of the present invention as it would be configured during use.

FIG. 5 is a perspective view of an alternative embodiment of the present invention comprising a reusable waist belt and releasably attachable suspension sling, similar to the view in FIG. 2.

### DETAILED DESCRIPTION OF THE PREFERRED EMBODIMENTS

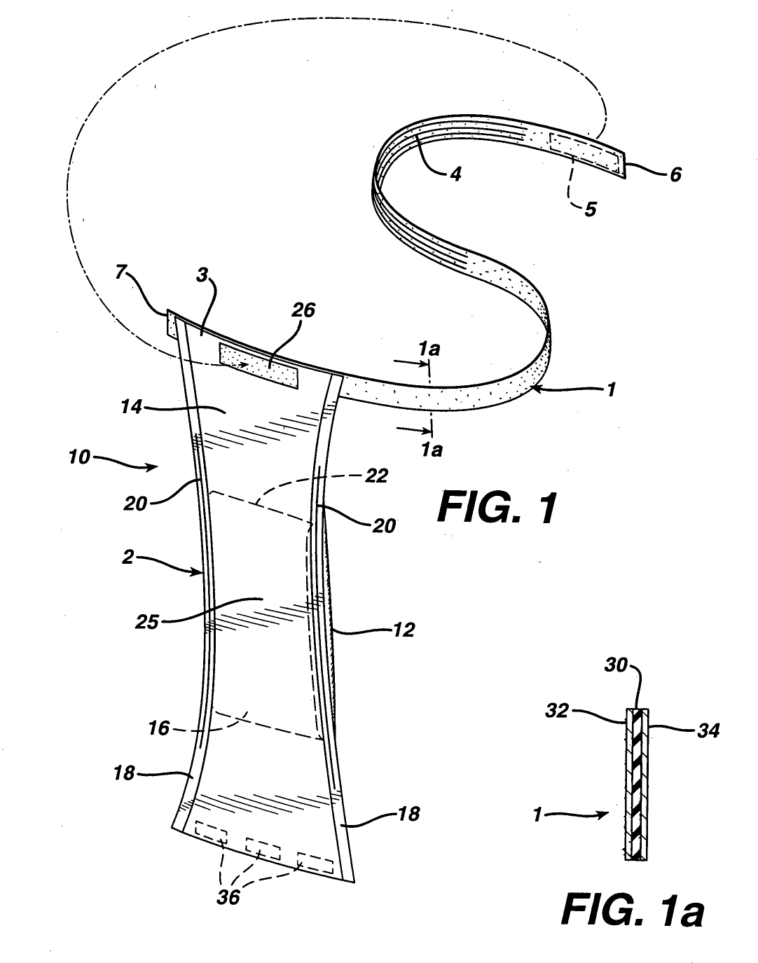

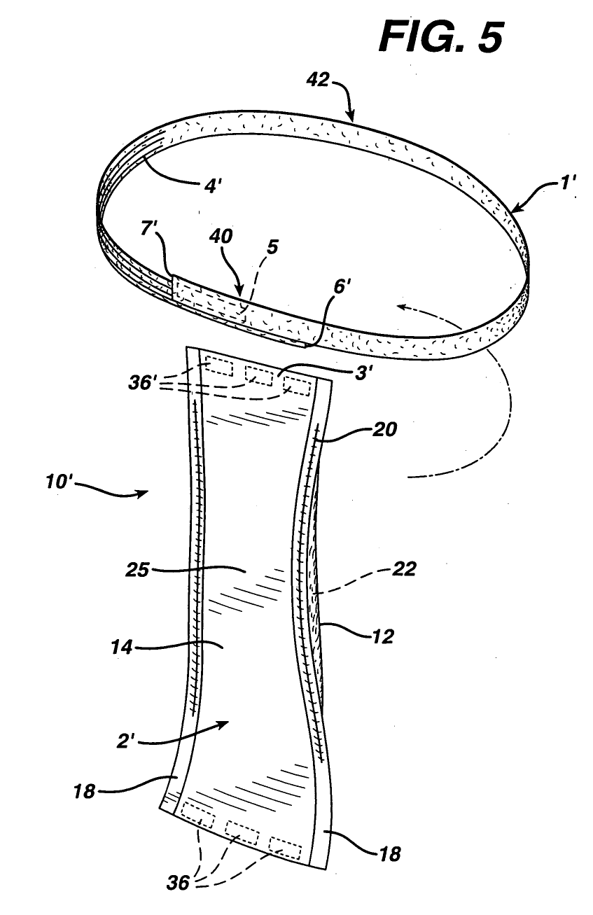

The present invention relates to absorbent garments such as an adult incontinence garment, and more particularly, to absorbent garments having an improved waist belt attachment system. In a first embodiment shown in FIGS. 1-4, the absorbent garment is a disposable product having an integral waist belt attachment system. In a second embodiment shown in FIG. 5, the absorbent garment comprises a reusable waist belt to secure a disposable absorbent suspension sling.

As used herein the specification and the claims, the term “integral disposable absorbent garment” refers to articles which absorb and contain body exudates and more specifically refers to articles which are placed against or in proximity to the body of a wearer to absorb and contain the various exudates discharged from the body and which are intended to be discarded after a single use (i.e., they are not intended to be laundered or otherwise restored or reused), and which are unitary in that they do not require separate manipulative parts like a separate holder and liner. A preferred embodiment of the integral disposable absorbent garment of the present invention is shown in FIG. 1.

As used herein the specification and the claims, the term “absorbent garment” refers to a garment generally worn by infants or other incontinent persons, or by menstruating females about the lower torso.

FIG. 1 is a perspective view of the garment 10 of the present invention in its flat out, uncontracted state (i.e., with all elastic induced contraction pulled out). As can be seen in FIG. 1, the garment 10 basically comprises an integral waist belt 1 attached to suspension sling 2 at the proximal end 3 of suspension sling 2. The integral waist belt 1 further comprises elastic elements 4 and a belt closure system 5 for use in attaching the second end 6 of the belt to the first end 7 of the belt. The suspension sling 2 may be any element that is generally compressible, conformable, non-irritating to the wearer's skin, and capable of absorbing and containing body exudates, such as feces, urine, blood, pus, and the like. In the preferred embodiment shown in FIG. 1, the suspension sling 2 comprises a liquid pervious topsheet 12; a liquid impervious backsheet 14; and an absorbent structure 16 that is positioned between said topsheet 12 and said backsheet 14.

Joined to the proximal end 3 of suspension sling 2 is the integral belt 1. The integral belt 1 is an elongated member or combination of members which are generally conformable and non-irritating to the wearer's skin and which serve to hold or suspend the garment about the wearer's lower torso. As used herein the specification and the claims, the term “joined” includes any means for affixing the members together. It includes, without limitation, (a) embodiments wherein the first member, (e.g., the integral belt 1) is a separate member constructed from the same or different material as the second member (e.g., an element of the suspension sling 2) and directly or indirectly secured to the second member (i.e., integral), and (b) embodiments wherein the first member is constructed from the same material as the second member in such a way that the first member comprises continuous and undivided elements of the second member (i.e., unitary). In the preferred embodiment shown in FIG. 1, the integral belt 1 is an elasticized nonwoven.

As used herein the specification and the claims, the term “attach” and variants thereof include the above defined “joined” and also means for affixing members together wherein the members can be removed without destroying at least one of the members.

The suspension sling 2 has a liquid receiving top surface that is generally defined by the topsheet 12 and a back surface that is generally defined by the backsheet 14. Preferably, the topsheet 12 and the backsheet 14 have length and width dimensions generally larger than the absorbent structure 16, so that they extend beyond the edges of the absorbent structure 16 where they are associated together in a suitable manner. Alternatively, the topsheet 12 and/or the backsheet 14 may have length and width dimensions generally larger than the absorbent structure 16 so that they wrap around the side edges of the absorbent structure 16 and fasten together under absorbent structure 16 to enclose absorbent structure 16. As used herein the specification and the claims, the term “associated” encompasses configurations in which the topsheet 12 is directly joined to the backsheet 14 by affixing the topsheet 12 directly to the backsheet 14 and configurations in which the topsheet 12 is indirectly joined to the backsheet 14 by affixing the topsheet 12 to intermediate members which in turn are affixed to the backsheet 14. In the preferred embodiment shown in FIG. 1, the extension of the topsheet 12 and/or the backsheet 14 beyond the side edges of the absorbent structure 16 forms the suspension sling side edges 18. It is preferred that the suspension sling side edges 18 are provided with side elastic elements 20 although these are not necessary. The elastic elements 20 may extend the entire length of the suspension sling side edges 18, or the elastic elements 20 may extend only along a portion of the length of the suspension sling side edges 18.

Examining some of the elements of the garment 10 in more detail, the topsheet 12 is positioned adjacent the body surface 22 of the absorbent structure 16 and overlies a major portion of the absorbent structure 16, so that when exudates are discharged onto the topsheet 12 they penetrate through the topsheet 12 where they are absorbed by the absorbent structure 16. The topsheet 12 is compliant, soft feeling, and non-irritating to the wearer's skin. Further, the topsheet 12 is liquid 25, pervious, permitting liquids to readily penetrate through its thickness. A suitable topsheet may be manufactured from a wide range of materials, such as woven fabrics, nonwoven fabrics, plastic nets, porous foams, reticulated foams, apertured plastic films, natural fibers (e.g., wood or cotton fibers), synthetic fibers (e.g., polyester of polypropylene fibers) or from a combination of natural and synthetic fibers. Preferably, it is made of hydrophobic material to isolate the wearer's skin from liquids in the absorbent structure 16. A particularly preferred topsheet 12 comprises a nonwoven comprising a blend of staple length polypropylene fibers having deniers of about 3 and about 5, such as that currently used as the topsheet on STAYFREE® Maxipads with Cottony Dry Cover, which are sold by Personal Products Company, Skillman, N.J.

There are a number of manufacturing techniques which may be used to manufacture the topsheet 12. For example, a fibrous topsheet 12 may be woven, non-woven, spunbonded, carded, or the like. Alternatively, the topsheet 12 may comprise a plastic net material such as that described in DeRossett et al., U.S. Pat. No. 4,710,186, or an apertured plastic film such as that made by the process described in Turi et al., U.S. Pat. No. 5,567,376. A preferred tcpsheet 12 is a nonwoven fabric formed by carding and thermally bonding fibers by means well known to those skilled in the fabrics art.

The absorbent structure 16 may be any element that is generally compressible, conformable, non-irritating to the wearer's skin, and capable of absorbing and containing liquids and certain body exudates (e.g., menses and/or urine). The absorbent structure 16 may be manufactured in a wide variety of sizes and shapes (e.g., rectangular, hourglass, etc.) and from a wide variety of liquid absorbent materials commonly used in disposable garments and other absorbent articles. A representative, non-limiting list of useful materials includes cellulosic materials, such as rayon, cotton, wood pulp, creped cellulose wadding, tissue wraps and laminates, Feat moss, and chemically stiffened, modified, or cross-linked cellulosic fibers; synthetic materials, such as polyester fibers, polyolefin fibers, absorbent foams, absorbent sponges, superabsorbent polymers, aborbent gelling materials; formed fibers, such as capillary channel fibers and multilimbed fibers; combinations of materials, such as synthetic fibers and wood pulp including coformed fibrous structures (e.g., those materials described in Anderson et al., U.S. Pat. No. 4,100,324); or any equivalent material or combinations of materials, or mixtures of these. The total absorbent capacity of the absorbent structure 16 should, however, be compatible with the designed exudate loading for he intended use of the garment 10. Further, the size and absorbent capacity of the absorbent stricture 16 may be varied to accommodate wearers ranging from infants to adults.

An exemplary embodiment of the garment 10 has an hourglass shaped absorbent structure 16 and is intended to be worn by adults. The absorbent structure 16 of the example is a batt of pulp fluff about 16 centimeters (6.4 inches) wide (lateral dimension along the core waist edges 54), about 45 centimeters (18.0 inches) long (longitudinal dimension) and about 11 centimeters (4.4 inches) across (lateral dimension) the narrowest part of the crotch region 25. It should be understood, however, that the size, shape, configuration, and total absorbent capacity of he absorbent structure 16 may be varied to accommodate wearers ranging from infants through adults. Therefore, he dimensions, shape, and configuration of the absorbent structure 16 may be varied (e.g., the absorbent layer may have a varying caliper, or a hydrophilic gradient or may contain absorbent gelling materials).

The backsheet 14 is positioned adjacent the garment surface of the absorbent structure 16 and is preferably attached thereto by attachment means (not shown) such as those well known in the art. For example, the backsheet 14 may be secured to the absorbent structure 16 by a uniform continuous layer of adhesive, a patterned layer of adhesive, or an array of separate lines or spots of adhesive. An adhesive that has been found to be satisfactory has been manufactured by the H. B. Fuller Company of St. Paul, Minn., under the name Fuller H L 1491 XPZ pressure sensitive adhesive.

The backsheet 14 is impervious to liquids and is preferably manufactured from a thin plastic film, although other flexible liquid impervious materials may also be used. The backsheet 14 prevents the exudates absorbed and contained in the absorbent structure 16 from wetting articles that contact the garment 10, such as bedsheets and undergarments. Preferably, the backsheet 14 is a polyethylene film having a thickness of from about 0.012 millimeters (0.5 mil) to about 0.051 centimeters (2.0 mils), although other flexible, liquid impervious materials may be used.

A suitable polyethylene film is a 1.0 mil thick film manufactured by Huntsman Packaging Corporation of Salt Lake City, Utah. The backsheet 14 is preferably embossed and/or matte finished to provide a more cloth-like appearance. Further, the backsheet 14 may permit vapors to escape from the absorbent structure 16 while still preventing liquid exudates from passing through the backsheet 14.

The size of the backsheet 14 and/or the topsheet 12 are dictated by the size of the absorbent structure 16 and the exact garment design selected. In a preferred embodiment, the backsheet 14 and the topsheet 12 have a substantially hourglass shape and extend beyond the side edges of the absorbent structure 16 a distance of from about 2.5 centimeters (1.0 inch) to about 8.75 centimeters 3.5 inches) to form extensions around the side edges of the absorbent structure. These extensions are joined together to enclose effectively the absorbent structure between the backsheet and the topsheet.

The first end 7 of the integral belt 1 is joined to the suspension sling 2 along the suspension sling proximal end 3 by fixed attachment points or by continuous attachment along the entire proximal end 3. The first end 7 of the integral belt 1 can be joined to the suspension sling 2 in a number of ways and at a number of points on the suspension sling 2. The integral waist belt 1 has a second end 6 which is free from the suspension sing 2 and which extends away from the suspension sing 2. In use, a part of the second end 6 is associated with either the proximal end 3 of suspension sing 2 or the first belt end 7 to fasten the garment 10 about the waist of the wearer.

The belt closure system 5 may include a first element joined to the suspension sling proximal end 3 and a second element joined to the second belt end 6 in order to fasten the waist belt 1 around the waist of a user. The belt closure system elements are provided to receive and/or hold each other, and they may, in association with the suspension sling 2, hold or suspend the suspension sling 2 about the lower torso of the wearer. In the preferred embodiment shown in FIG. 1, one belt closure system element 5 is joined to the backsheet 14 of the suspension sling proximal end 3 and another belt closure system element 5 is joined to the second belt end 6. In this preferred embodiment, the belt closure system elements 5 comprise at least one adhesive material, such as pressure sensitive adhesive or a cohesive adhesive system. Preferably, the adhesive material is a segment of double-faced adhesive tape. Other belt closure systems are available. For instance, the belt closure system may utilize a pair of belt loops through which the second belt end 6 can be brought and then fastened to hold it in place. The belt loops can be separate members that are attached to the backsheet 14 of the suspension sling proximal end 3.

Alternatively, the belt loops can be formed by cutting slits in the backsheet 14 through which the single second belt end 6 can be brought. Other alternative belt closure systems include mechanical fasteners. As used herein die specification and the claims, the term “mechanical fastener” refers to fastening systems that releasably join together through mechanical interaction. A representative, non-limiting list of such fasteners includes hook-and-loop type fasteners (e.g., VELCRO brand fasteners), snaps, or hooks that interact with other elements of the belt closure system 5. An example of such hooks is described in U.S. Pat. No. 5,230,851 to Thomas et al., and an example of a nonwoven to which such hooks may be mechanically attached is described in Goulait, U.S. Pat. No. 5,407,439.

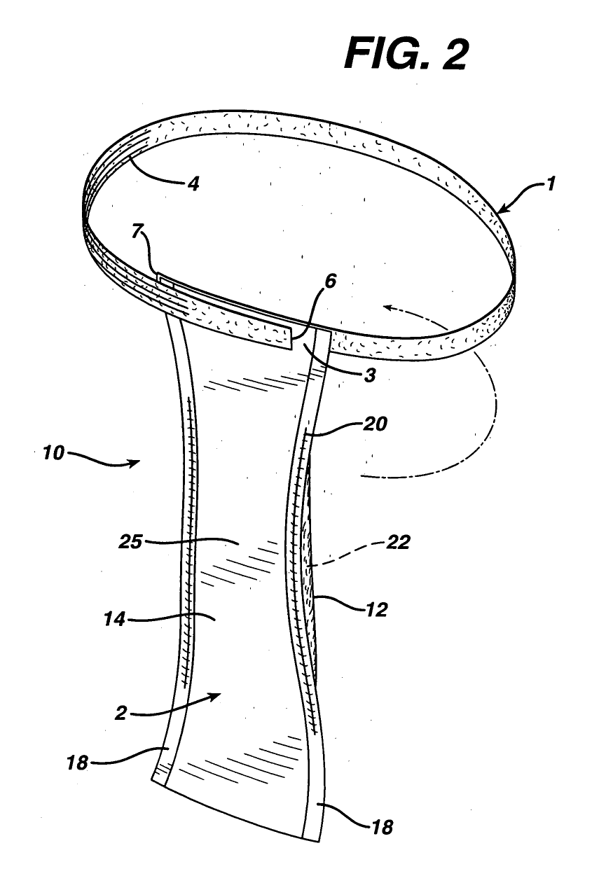

As shown in FIG. 2, belt closure system interacts to secure the waist belt 1 around the waist of a wearer. The belt closure system 5 is intended to hold the waist belt 1 about the waist of a wearer and to allow for the suspension of the suspension sling 2 about the lower torso of the wearer.

It is preferred that the waist belt 1 be a completely separate member from the suspension sling 2 which has been sewn, adhered, or joined by any other means to the suspension sling 2. It is also preferred that the waist belt 1 be elastically contractible. A preferred elastically contractible waist belt 1 is a laminate of a pre-stretched elastic member 30 between two webs 32 and 34, such as hat shown in FIG. 1A. Webs 32 and 34, which may be similar or different materials, may be woven, knit, or nonwoven fabrics, films, apertured films, or plastic mesh or netting. Elastic member 30 may comprise, without limitation, elastic film, elastic nonwoven, stretchable woven or knit fabric, elastic strands, elastic foam, elastic adhesive, or a combination of these.

Alternatively, the waist belt 1 may be an extension of the topsheet and/or the backsheet and integral with the suspension sling 2.

In the alternative embodiment illustrated in FIG. 5, the reference numerals of FIGS. 1-4 are repeated with the alteration that changed elements are reflected by the use of the “′” notation. Thus, the waist belt of the alternative embodiment is referred to by the reference numeral 1′. Thus, the second belt end 6′ may be releasably attached to the first belt end 7′ by means of a belt closure system 5′. In addition, the suspension sling proximal end 3′ may be attached to a first sling attachment location 40 on the waist belt 1′ proximate the first belt end 7′. Preferably, this attachment occurs after the first belt end 7′ and second belt end 6′ are attached together to reduce the likelihood of any significant distortion of the suspension sling 2′ due to subsequent distortion of the waist band 1′. In addition, the suspension sling distal end 38 may be attached to a second sling attachment location 42 on the waist belt 1′. The second sling attachment location 42 is disposed between the first belt end 7′ and second belt end 6′, preferably about midway between the two ends.

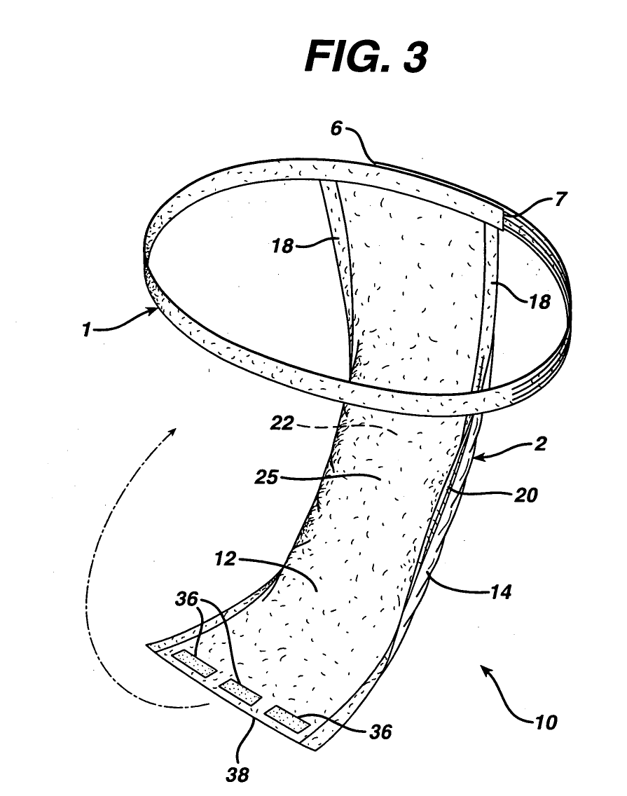

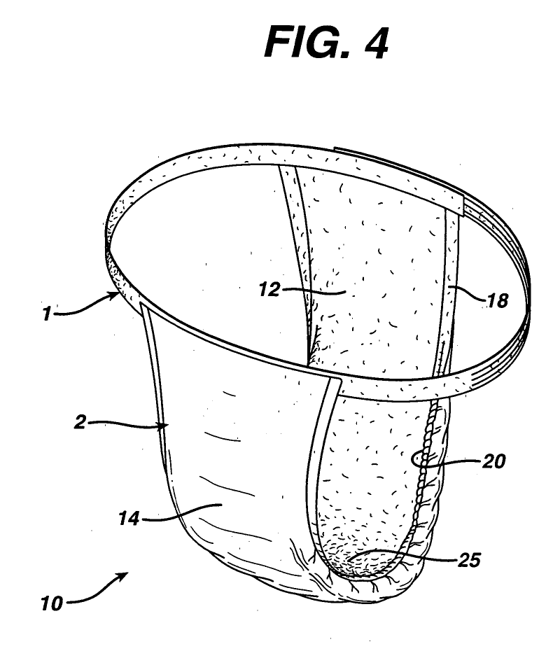

A brief description of the use of the integral disposable absorbent product referring to FIGS. 1-4 follows. First, the wearer may wrap the waist belt 1 around her waist and fasten the belt closure system 5 together in such a way that the waist belt 1 is securely and comfortably fastened around the wearer's waist and suspension sing 2 is suspended generally in the front of the wearer, as shown in FIG. 2. At this time, the wearer would be able to easily make adjustments to the belt closure system 5 to assure that the waist belt 1 is comfortably and securely fastened about her waist. The wearer then may rotate the product around her waist so that the suspension sling is hanging generally over her buttocks (not shown). The suspension sling then may be pulled forward between the wearer's legs, as shown in FIG. 3. The suspension sling fasteners 36 then may attach the suspension sling distal end 38 to the waist belt 1. FIG. 4 shows the garment as it would appear when in place about a wearer's lower torso.

Examples of suitable suspension sling fasteners 36 may be, without limitation, pressure sensitive adhesives, mechanical fasteners, cohesive adhesives, or any other fastening material that is capable of interacting with waist belt 1 to secure the suspension sling distal end 38 to the waist belt.

In the embodiment illustrated in FIG. 5, the above method could be employed to use the device, with the additional step that the suspension sling proximal end 3′ would have to be attached to the waist belt 1′, preferably at the first sling attachment location 40. As suggested above, this may most advantageously be done after the belt closure system 5 has been engaged to attach the first belt end 7′ to the second belt end 6′. The remaining steps described with reference to FIGS. 1-4 could then be followed. Of course, it is also possible to attach the suspension sling proximal end 3′ to the first sling attachment zone 40 prior to fastening the waist belt 1′ about the user's torso. This method would be substantially identical to that described above for FIGS. 1-4.

While particular embodiments of the present invention have been illustrated and described, it would be obvious to those skilled in the art that various other changes and modifications can be made without departing from the spirit and scope of the invention.

What is claimed is:

1. An integral disposable absorbent garment that may be worn about a human lower torso, said garment comprising:
a) a suspension sling for absorbing and containing body exudates, said suspension sling having longitudinally extending suspension sling side margins, a suspension sling distal end and a suspension sling proximal end, said suspension sling being suspendable in a wearer's crotch region; and
b) a waist belt having a first belt end secured to the suspension sling proximal end and a second belt end;
wherein the waist belt is capable of encircling a user's waist with the suspension sling suspended in the wearer's crotch region.

2. The integral disposable absorbent garment as claimed in claim 1, wherein said suspension sling comprises:
a) a liquid permeable topsheet;
b) a liquid impermeable backsheet associated with said topsheet; and
c) an absorbent structure positioned between said topsheet and said backsheet.

3. The integral disposable absorbent garment as claimed in claim 2 wherein the suspension sling further comprises an elastic element.

4. The integral disposable absorbent garment as claimed in claim 3 wherein the elastic element is disposed in at least one suspension sling side margin.

5. The integral disposable absorbent garment as claimed in claim 1, wherein said waist belt is elastically contractible.

6. The integral disposable absorbent garment as claimed in claim 1, further comprising a belt attachment system.

7. The integral disposable absorbent garment as claimed in claim 6 wherein the belt attachment system is a pressure sensitive adhesive.

8. The integral disposable absorbent garment as claimed in claim 6 wherein the belt attachment system is a mechanical fastener.

9. The integral disposable absorbent garment as claimed in claim 6, wherein said suspension sling distal end further comprises suspension sling fasteners.

10. A method of applying an absorbent undergarment about a human lower torso having a waist and a crotch comprising the steps of:
a) encircling the waist of the torso with a waist belt comprising a first belt end secured to a proximal end of a suspension sling and a second belt end;
b) releasably attaching the second belt end to the first belt end;
c) drawing the suspension sling through the crotch of the torso, the suspension sling further comprising means for absorbing and containing body exudates, longitudinally extending suspension sling side margins, and a suspension sling distal end; and
d) releasably attaching the suspension sling distal end to the belt at a point between the second belt end and the first belt end.

11. The method of claim 10 further comprising orienting the second belt end and suspension sling toward the anterior of the torso while releasably attaching to the first belt end thereto.

12. The method of claim 11 further comprising rotating the encircling belt about the waist to locate the suspension sling at posterior portions of the torso, generally in the area of the buttocks, prior to drawing the distal end through the crotch toward anterior portions of the torso.

13. An absorbent garment that may be worn about a human lower torso, said garment comprising:
a) a waist belt having a first belt end, a second belt end, a first sling attachment location proximate the first belt end, and a second sling attachment location disposed between the first and second belt ends; and
b) a disposable suspension sling for absorbing and containing body exudates, said suspension sling having longitudinally extending suspension sling side margins, a suspension sling distal end and a suspension sling proximal end, said suspension sling being attachable to the first and second sling attachment locations of the waist belt in a manner to suspend the suspension sling in a wearer's crotch region;
wherein the waist belt is capable of encircling a user's waist with the suspension sling suspended in the wearer's crotch region.

14. The absorbent garment of claim 13 wherein the second sling attachment location is disposed approximately midway between the first and second belt ends.

15. The absorbent garment of claim 13 wherein the waist belt and suspension sling comprise a belt closure system.

16. The absorbent garment of claim 15 wherein the belt closure system comprises closure system elements at least at the first and second sling attachment locations and proximate the suspension sling distal and proximal ends.

17. The absorbent garment of claim 16 wherein closure system elements join the first sling attachment location to the suspension sling proximal end.

18. A method of applying an absorbent undergarment about a human lower torso having a waist and a crotch comprising the steps of:
a) encircling the waist of the torso with a waist belt having a first belt end, a second belt end, a first sling attachment location proximate the first belt end, and a second sling attachment location disposed between the first and second belt ends;
b) releasably attaching the first and second belt ends;
c) attaching to the first sling attachment location a proximal end of a disposable suspension sling useful for absorbing and containing body exudates, said suspension sling having longitudinally extending suspension sling side margins, a suspension sling distal end and the suspension sling proximal end;
d) drawing at least the suspension sling distal end through the crotch of the torso; and
e) attaching the suspension sling distal end to the second sling attachment location.

19. The method of claim 18 further comprising the step of orienting the first belt end toward anterior portions of the torso while releasably attaching to the second belt end thereto.

20. The method of claim 19 further comprising the steps of attaching the suspension sling proximal end to the first sling attachment location while the first and second belt ends are oriented toward the anterior of the torso and rotating the belt about the waist to locate the suspension sling adjacent posterior portions of the torso, generally in the area of the buttocks, prior to drawing the distal end through the crotch toward the anterior portions of the torso.
